SEQUENCE LISTING (17 sequences)

SEQ ID NO: 1 - RNA, 11 nt, Artificial
  telomerase RNA fragment
cuaacccuaac

SEQ ID NO: 2 - DNA, 545 bp, Homo sapiens
gagtgactctcacgagagccgcgagagtcagcttggccaatccgtgcggtcggcggccgctccctttataagccgactcgcccggcagcgcaccgggttgcggagggtgggcctgggaggggtggtggccattttttgtctaaccctaactgagaagggcgtaggcgccgtgcttttgctccccgcgcgctgtttttctcgctgactttcagcgggcggaaaagcctcggcctgccgccttccaccgttcattctagagcaaacaaaaaatgtcagctgctggcccgttcgcccctcccggggacctgcggcgggtcgcctgcccagcccccgaaccccgcctggaggccgcggtcggcccggggcttctccggaggcacccactgccaccgcgaagagttgggctctgtcagccgcgggtctctcgggggcgagggcgaggttcaggcctttcaggccgcaggaagaggaacggagcgagtccccgcgcgcggcgcgattccctgagctgtgggacgtgcacccaggactcggctcacacatgc

SEQ ID NO: 3 - DNA, 4015 bp, Homo sapiens
gcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 4 - protein, 1132 aa, Homo sapiens
MPRAPRCRAVRSLLRSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 5 - RNA, 11 nt, Homo sapiens
caaucccaauc

SEQ ID NO: 6 - DNA, 21 bp, Artificial
  oligonucleotide
uugucuaacccuaacugagtt

SEQ ID NO: 7 - DNA, 21 bp, Artificial
  oligonucleotide
cucaguuaggguuagacaatt

SEQ ID NO: 8 - DNA, 21 bp, Artificial
  oligonucleotide
ggcttctccggaggcaccctt

SEQ ID NO: 9 - DNA, 21 bp, Artificial
  oligonucleotide
gggtgcctccggagaagcctt

SEQ ID NO: 10 - DNA, 21 bp, Artificial
  oligonucleotide
caagguggaugugacgggctt

SEQ ID NO: 11 - DNA, 21 bp, Artificial
  oligonucleotide
gcccgucacauccacguugtt

SEQ ID NO: 12 - DNA, 22 bp, Artificial
  primer
ctgggaggggtggtggccattt

SEQ ID NO: 13 - DNA, 22 bp, Artificial
  primer
cgaacgggccagcagctgacat

SEQ ID NO: 14 - DNA, 23 bp, Artificial
  primer
gccagaacgttccgcagagaaaa

SEQ ID NO: 15 - DNA, 23 bp, Artificial
  primer
aatcatccaccaaacgcaggagc

SEQ ID NO: 16 - DNA, 16 bp, Homo sapiens
ccctaaccctaaccct

SEQ ID NO: 17 - DNA, 26 bp, Homo sapiens
agggttagggttagggttagggttag

(19) United States
(12) Patent Application Publication
(10) Pub. No.: US 2005/0003404 A1
(43) Pub. Date: Jan. 6, 2005

(54) Telomerase interference

(76) Inventor: Peter T. Rowley, Rochester, NY (US)

Correspondence Address: Richard F. Trecartin;DORSEY & WHITNEY LLP, Four Embarcadero Center, Suite 3400, San Francisco, CA, 94111-4187, US

(21) Appl. No.: 10/831,266

(22) Filed: Apr. 22, 2004

Related U.S. Application Data
(63) Continuation of application No. PCT/US02/33065, filed on Oct. 16, 2002.
(60) Provisional application No. 60/359,196, filed on Feb. 20, 2002.
(60) Provisional application No. 60/383,195, filed on May 22, 2002.
(60) Provisional application No. 60/345,326, filed on Oct. 22, 2001.

(51) Int. Cl. C12Q 1/68; C07H 21/02; C12N 9/22

(52) U.S. Cl. 435/6; 536/23.1; 435/69.1; 435/199; 435/320.1; 435/325

(57) ABSTRACT

The invention relates to nucleic acids encoding or comprising interfering RNAs which target telomerase RNA or mRNA encoding the telomerase reverse transcriptase (TERT). The invention includes methods for inhibiting telomerase activity expression vectors, and pharmaceutical compositions.

### TECHNICAL FIELD

The invention is directed to nucleic acids and methods for interfering with telomerase activity using double stranded RNA.

### BACKGROUND

Telomerase is attracting increasing attention in cancer research because of the striking correlation of telomerase activity with malignancy. Telomerase activity is present in most malignant types of tumors, but absent in most normal somatic tissues. (Shay, Molec Med Today 1:378-384 (1995.) Among normal cells, activity is detectable only in embryonic cells, in adult male germline cells, and in proliferative cells of renewable tissues e.g. activated lymphocytes, hematopoietic stem cells, basal cells of the epidermis, and intestinal crypt cells. (Kim, et al., Science 266:2011-2015 (1994); Shay, et al., J Clin Pathol 50:106-109 (1997)). These normal cells having telomerase activity are expected to be less susceptible than malignant cells to telomerase inhibitors because normal cells generally have longer telomeres than do malignant cells and because those normal cells that are stem cells are generally quiescent i.e. in Go stage in which telomerase is not activated. (Kim, Europ. J Cancer 33:781-786 (1997).) DNA polymerase in higher organisms does not initiate the synthesis of a new DNA strand. Rather, it extends an existing partial strand, referred to as a primer, that is hybridized to the template. This results in an incomplete copy of the template strand. The primer, which is RNA, is destroyed at the end of replication, leaving the 5′-end of the new strand incomplete. Thus, successive rounds of DNA replication predictably lead to a progressive shortening of the DNA (Harley, J NIH Res 7:64-68 (1995)).

Telomerase provides one solution to this end-replication problem. It is a DNA polymerase that specializes in synthesizing DNA ends. Somatic cells generally lack detectable telomerase activity (Kim, et al., Science 266:2011-2015 (1994)). For this reason it has been suggested that the acquisition of telomerase activity is a necessary condition for a cell to acquire immortality (Harley, Mutat Res. 256:271-281 (1991)). After all, it is their immortality that makes cancer cells so dangerous to the organism. This hypothesis was confirmed by Bodnar et al. (Bodnar, et al., Science 279:349-352 (1998)) who demonstrated that normal human cells transfected with the gene for human telomerase catalytic subunit exceed their normal lifespan while maintaining a normal karyotype and youthful morphology.

A dominant negative mutant form of the catalytic subunit of human telomerase resulted in complete inhibition of telomerase activity, a reduction in telomere length, and death of tumor cells (Hahn, et al., Nature Med 5:1164-1170 (1999)). Further, in vivo expression of this mutant telomerase eliminated tumorigenicity. Since disruption of telomeric maintenance limits cellular lifespan in human cancer cells, telomerase is a promising target for anticancer therapy.

Chromosomes normally end in telomeres. The DNA sequence of telomeres is a repeated sequence of 5-8 nucleotides, rich in G, but differing among species. Human telomeres contain the 6-nucleotide sequence TTAGGG, repeated up to 15 kb (Allshire, et al., Nature 332:656-659 (1988); Moyzis, et al., Proc Natl Acad Sci USA 85:6622-6626 (1988); Morin, Cell 59:521-529 (1989)). Telomeres are essential to chromosomal integrity. Chromosomes lacking their normally constituted ends are unstable and fuse with other chromosomes or are lost when cells divide (Muller, Woods Hole 13:181-198 (1938); McClintock, Genetics 41:234-282 (1941)). Their specific sequence is presumed to mediate telomere function by binding specific proteins that protect it, i.e. by shielding the ends of chromosomes from reparative or degradative enzymes that might otherwise identify them as products of DNA breakage (de Lange, EMBO J 111:717-724 (1992)).

Tetrahymena was the first organism used to discover an activity that adds telomeric sequences to single stranded telomeric oligodeoxyribonucleotides, and to demonstrate that telomere synthesis requires a primer, but does not require DNA polymerase-alpha (Greider, et al., Cell 43:405-413 (1985)). Such telomerase activity is RNase-sensitive and therefore requires RNA, presumably as a template (Greider, Cell 51:887-898 (1987)). Finally, Tetrahymena telomerase RNA was cloned and was shown to contain a sequence complementary to the telomeric DNA repeat sequence (Greider, et al., Nature 337:331-337 (1989)). Thus, telomerase was shown to be a ribonucleoprotein with RNA-dependent DNA polymerase activity. The proposed model of its action requires that the enzyme add six nucleotides in a given location sequentially, then translocate distally to add the next six nucleotides.

Antisense technology utilizes single stranded DNAs or RNAs that are complementary to a single stranded target region which is usually an mRNA. Antisense nucleic acids interfere specifically with the target by forming base-pairing interactions. Formation of a double-strand can block the biological fuinction of the target. Various forms of antisense nucleic acids can be used. These include endogenously expressed antisense RNA or synthetic oligonucleotides, mostly DNA oligonucleotides. Synthetic oligonucleotides may carry a variety of chemical modifications that make them less sensitive to enzymatic degradation. Many of these chemical modifications have been used in developing antisense agents to inhibit telomerase activity.

In a review of telomerase inhibitors (Rowley, et al., Anticancer Res 20:4419-4430 (2000)) the findings of 29 reports using antisense and 4 reports using ribozymes are summarized. Published efforts to inhibit telomerase using antisense technology have been directed almost exclusively at telomerase RNA, and chiefly at its template region. Antisense agents have included in vivo generated antisense RNA (full or partial) or synthetic antisense DNA and RNA oligonucleotides, including those that carry chemical modifications such as phosphorothioates, methylphosphonates, and 2-O-methylated agents. Phosphorothioates and peptide nucleic acids have been more active than phosphodiesters. Concentrations in the low nanomolar range have sufficed for 50% inhibition of activity. However, some inhibition has been observed with control sequences. There have been fewer reports of ribozyrnes than of antisense agents. G-quadruplex-binding agents have attracted attention in part because of the prospect of elucidating structure-function relations, but are less active than nucleotide-sequence related compounds and their specificity for telomerase is in doubt. The activity of many of the other agents is no doubt indirect. Few studies have evaluated telomere shortening, perhaps the most important effect.

Recently, there have been reports using antisense against telomerase RNA and two using a ribozyme against hTERT. One reports that peptide nucleic acids and 2-O-methyl RNA oligomers against telomerase RNA can inhibit telomerase activity resulting in telomere shortening and eventually apoptosis. (Herbert BS, et al. Proc Nat Acad Sci 96:14276-14281, 1999.)

Due to the toll that cancer takes on human lives, there is a need to develop therapeutic methods for treatment of cancer. Inhibiting telomerase activity in immortal cells, such as cancer cells, leads to telomere shortening and death. Feng et al., Science 269: 1236-41 (1995) and U.S. Pat. No. 5,583,016 report that transfection of immortalized cell lines with expression vectors encoding hTR antisense transcripts resulted in telomere shortening and cell crisis, characterized by a marked inhibition of cell growth.

Accordingly, an object of the invention is to provide methods to inhibit telomerase in cells alone or as a complement to other cancer therapy using conventional agents.

A further object of the invention is the development of a nucleic acid capable of forming a double stranded RNA targeting telomerase.

Still further, it is an object of the invention to provide pharmaceutical compositions for treating cancer.

### SUMMARY OF THE INVENTION

The invention relates to the discovery that double stranded interfering RNAs which target telomerase RNA or mRNA encoding the telomerase reverse transcriptase (TERT) are capable of inhibiting telomerase activity. Such interfering RNA's include double stranded short interfering RNA's (siRNAs). The double stranded region of the siRNA preferably comprises less than 30 nucleotide base pairs.

The invention also includes methods for inhibiting telomerase activity comprising treating a telomerase expressing cell with the above nucleic acid, where the nucleic acid encodes or comprises a double stranded interfering RNA which targets telomerase RNA or mRNA encoding Telomerase Reverse Transcriptase (TERT). When telomerase RNA is targeted, the target sequence is, in one embodiment, the telomerase template sequence. Specific embodiments target the sequence CUAACCCUAAC.

When TERT mRNA is targeted, it is preferred that the target region corresponds to the wild type region which corresponds to the reported dominant negative mutation in TERT. This mutant sequence, in TERT, comprises GAUGUG.

The invention also includes a pharmaceutical composition comprising the above nucleic acid in combination with a pharmaceutically acceptable carrier.

### BRIEF DESCRIPTION OF THE DRAWINGS

FIG. 1 depicts the process involved in adding nucleotide repeats to 3′ chromosomal ends.

FIG. 2 shows the sequence of human telomerase RNA (Genbank Accession No. U86046.1).

FIG. 3 shows the sequence of human telomerase reverse transcriptase mRNA (Genbank Accession No. AF015950.1; coding sequence 56-3454).

FIG. 4 shows the amino acid sequence of telomerase reverse transcriptase (Genbank Accession No. AAC51672.1).

FIG. 5 demonstrates that siRNAs for hTR and hTERT depress the telomerase activity of HCT-15 human colon carcinoma cells in a dose-dependent manner at 44 h.

FIG. 6 demonstrates that the effect of siRNA targeting hTR and hTERT on HeLa human cervical carcinoma cells is dose-dependent at 42 h.

FIG. 7 demonstrates the effect of siRNA targeted to hTR and hTERT on cells of mesodermal origin, viz. HT-1080 human fibrosarcoma cells.

FIG. 8 shows a comparison of siRNAs targeting two different sites in hTR for telomerase activity. HeLa cells were transfected with siRNA for hTR at various concentrations, assayed at 27 and 51 h. Solid bars represent hTR#1; hatched bars represent hTR#2 siRNA.

### DETAILED DESCRIPTION

The invention provides nucleic acids encoding or comprising sense and antisense nucleic acids which are capable of forming double stranded RNAs that inhibit telomerase and methods using such nucleic acids to inhibit telomerase activity in cells.

Telomerase is a target for inhibition in cancer and germline cells where telomerase is responsible for their immortality. Double stranded interfering RNA is used to inhibit telomerase because of its target specificity, its greater effectiveness than antisense nucleic acids and its applicability across species. Short double stranded interfering RNA is used presumably to interfere with telomerase because it avoids induction of an undesired interferon response.

As used herein, the term “telomerase” refers to an eukaryotic enzyme which comprises a telomerase reverse transcriptase (TERT) subunit and telomerase RNA (TR). Telomerase is a DNA polymerase that specializes in synthesizing DNA at the ends of chromosomes which contain telomeres. The telomere DNA sequence is a repeat sequence of 5 to 8 nucleotides rich in G but differing amongst species. Human telomeres contain the six-nucleotide sequence TTAGGG, repeated up to 15 kb (Allshire, et al., Nature 332:656-659 (1988); Moyzis, et al., Proc Natl Acad Sci USA 85:6622-6626 (1988); Morin, Cell 59:521-529 (1989)).

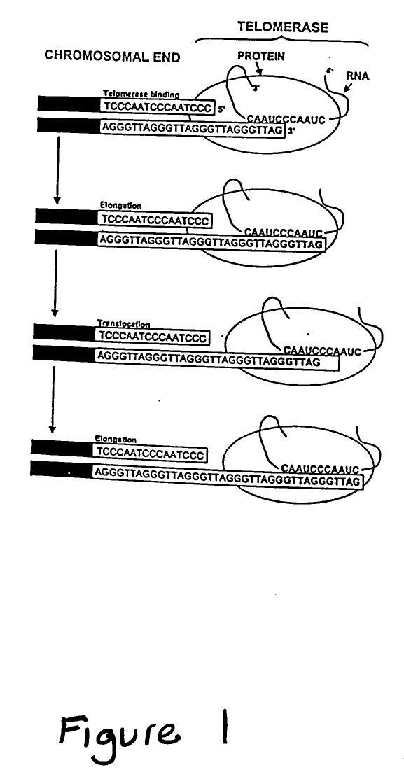

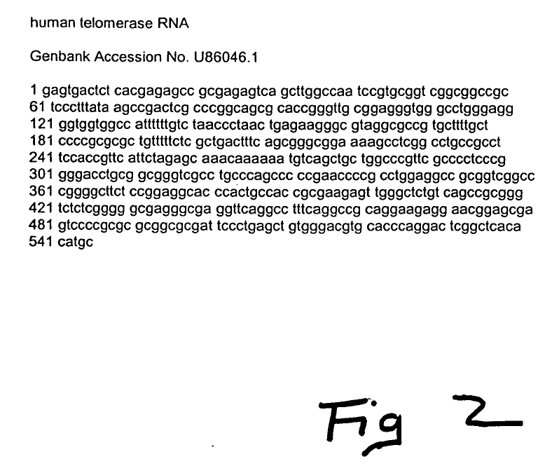

FIG. 1 depicts the process involved in adding the six nucleotide repeats to 3′ chromosomal ends. As can be seen, a portion of the telomerase RNA of the catalytic subunit of TERT hybridizes to the 3′ end of the telomeres. The elongation of the telomeres occurs by way of the reverse transcriptase activity of the telomerase to add the sequence GGTTAG. This is the same as the sequence TTAGGG except for being viewed in a different reading frame. Translocation may then occur which results in a shifting of the telomerase to the end of the newly added repeat followed by further elongation. Thus, telomerase can add one or more telomeric repeating units to the 3′ end of chromosomes.

Telomerase RNA (TR) refers to a nucleic acid encoding the RNA found in telomerase. The sequence for human TR (hTR) is set forth in FIG. 2 and can be found in Genbank Accession No. U86046. That portion of hTR sequence which binds to an accessible telomere and which provides a template for elongation of the telomere can be found between residues 48 and 60 and corresponds to the sequence CAAUCCCAAUC. The binding portion of this sequence corresponds to CAAUC. The elongation sequence corresponds to CCAAUC. The binding portion and elongation portion of telomerase RNA defines the telomerase template sequence. The human telomerase template sequence is common among most vertebrates.

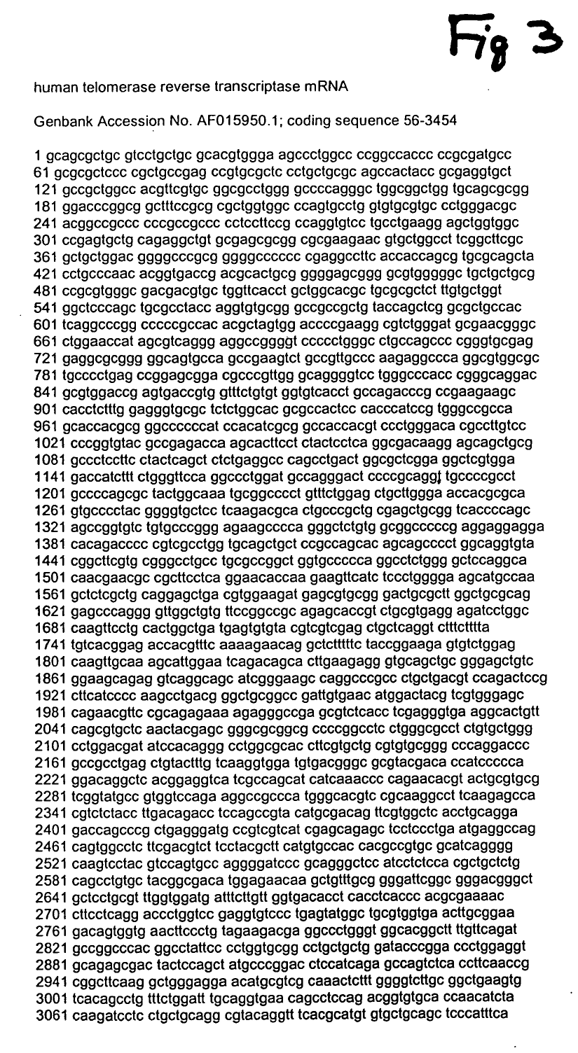

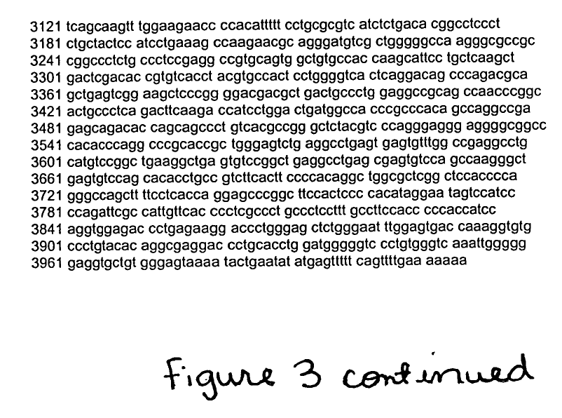

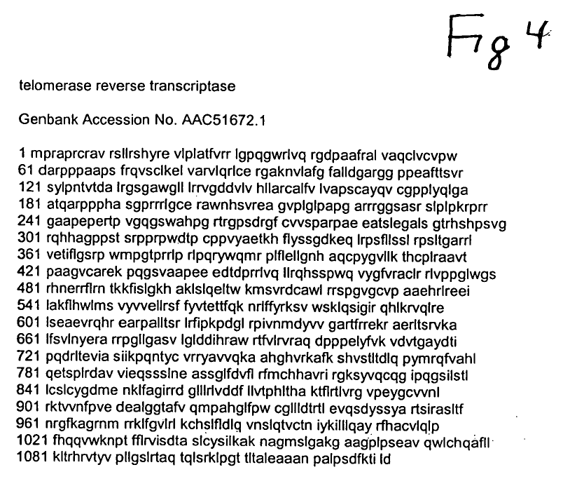

The DNA sequence encoding the catalytic subunit of human telomerase reverse transcriptase (hTERT) is set forth in FIG. 3 and corresponds to Genbank Accession No. AF0159050.1. The protein sequence for the catalytic subunit is set forth in FIG. 4.

As used herein, a double stranded interfering RNA refers to a composition of matter which contains a region having a double stranded RNA sequence. The double stranded region comprises “sense” and “antisense” RNA strands which are capable of hybridizing to each other. Alternatively, such sense and antisense strands may be covalently linked to each other by way of a linker which may be RNA transcribed from a DNA expression cassette with the sense and antisense regions of the transcribed RNA forming double stranded RNA. Other convenient linkers which provide for the capability of the sense and antisense strands to form a double stranded RNA may be used. For example, when the double stranded RNA is made synthetically, residues of hexaethylglycol (HGG) may be incorporated into the linker segment during standard solid phase synthesis. See G Jaschke, et al., Tetrahedron Lett. 34, 301 (1993). The HGG residues serve to reduce the number of synthetic steps required to span the ends of the sense and antisense strands which form the double stranded interfering RNA. This method is particularly useful when the interfering RNA contains one or more deoxyribonucleotides at the ends of the sense and antisense RNAs so as to provide a convenient point of covalent attachment to the linker.

Because of the interferon response which may be induced by long double-stranded RNA's, it is preferred that the double stranded interfering RNA comprise a short double stranded region. Such RNAs are referred to as short interfering RNA's (siRNA).

The double stranded siRNA in general will have a double stranded region having no more than about 40 nucleotide base pairs, more preferably no more than about 30 nucleotide base pairs, more preferably no more than about 25 nucleotide base pairs, and preferably no more than about 19 nucleotide base pairs. As such, the preferred range of double stranded region in an siRNA is between 19 and 40 nucleotide base pairs, more preferably between 19 and 30 nucleotide base pairs, and most preferably between 19 and 25 nucleotide base pairs.

For double stranded interfering RNA's other than siRNA, the length of the double stranded RNA region can be as long as the length of the mRNA encoding TERT, i.e., about 3400 nucleotide base pairs or the length of the telomerase RNA, i.e., about 546 nucleotide base pairs. Smaller lengths are preferable and can be approximately 450-500 nucleotide base pairs and as low as about 40 nucleotide base pairs.

The mode of action of the double stranded interfering RNA is believed to involve one or more enzymes which process the double stranded interfering RNA into a form which is capable of interacting with mRNA's or other single stranded RNA's so as to facilitate their enzymatic degradation. Accordingly, the double stranded interfering RNA is chosen so that it corresponds to a specific sequence within the single stranded RNA being targeted.

An interfering RNA corresponds to a single stranded target RNA if one of the sense or antisense strands in the double stranded region is complementary to or substantially complementary to all or a portion of the target RNA. Substantial complementing can be determined by sequence comparison to the target RNA. The interfering RNA is substantially complementary to the target RNA when the sense and antisense strand comprises no more than one or two substitutions over 20 nucleotides as compared to the opposite strand or the target sequence. It is preferred that the antisense strand be identical to the target sequence.

In a preferred embodiment, the double stranded interfering RNA is targeted to the telomerase RNA of the catalytic subunit of telomerase (e.g., hTR). More particularly, a double stranded siRNA is targeted to the telomere template sequence CUAACCCUAAC.

The double stranded siRNA targeting the aforementioned CUAACCCUAAC region of telomerase RNA may contain additional nucleotides both 5′ and 3′ to the RNA and in some embodiments complements nucleotides on the opposing strand. Additional nucleotides are in general chosen to fuirther the hybridization and therefore the targeting of the double stranded siRNA. In some embodiments, it is preferred that at least one of the ends of the double stranded siRNA contain one or more additional 3′ nucleotides so as to form an overhanging region. This overhanging region is preferably two unpaired nucleotides at the 3′ termini. If present, the overhang may be complementary to the target and can be a ribonucleotide and or deoxiribonucleotide particularly two thymidine deoxynucleotides. An example of a double stranded siRNA targeting the repeat template sequence mhTR is set forth as SEQ ID NO:1 where the bold nucleotide corresponds to the telomerase template sequence.

5′-UUGU CUA ACC CUA ACU GAG-TT-3′
3′-TT-AACA GAU UGG GAU UGA CUC-5′.

A second example of a specific siRNA targeting the telomerase RNA corresponds to the sequence in SEQ ID NO:2

5′-GGCT TCT CCG GAG GCA CCC TT-3′
3′-TT-CCGA AGA GGC CTC CGT GGG-5′.

This particular double stranded siRNA targets a 19 base pair sequence centered in the 26 base bp L loop which corresponds to the longest single stranded region in hTR according to the secondary structure proposed by Jen, et al., Cell 100:503-514 (2000).

In an alternative embodiment, a double stranded siRNA targets the mRNA encoding hTERT. Such targeting of hTERT mRNA can be alone or in combination with targeting of the telomerase RNA. An siRNA for targeting hTERT mRNA is

5′-CAAG GUG GAU GUG ACG GGC TT-3′
3′-TT-GUUG CAC CUA CAC UGC CCG-5′

This invention provides methods of interfering with telomerase activity by contacting the target RNA in vivo with the interfering nucleic acid of the invention. In cells, interference of telomerase activity renders an immortal cell mortal. Telomerase interference therapy is expected to be useful against cancers involving uncontrolled growth of immortal cells. Delivery of interfering nucleic acids against the target RNA of telomerase prevents telomerase action and ultimately leads to cell senescence and cell death.

In one method of the invention, telomerase interference involves contacting telomerase with an interfering nucleic acid directed against the target region of the telomerase.

By “nucleic acid” or “oligonucleotide” or grammatical equivalents herein means at least two nucleotides covalently linked together. A nucleic acid of the present invention will generally contain phosphodiester bonds, although in some cases, nucleic acid analogs are included that may have alternate backbones which, if used, are preferably used to link sense and antisense nucleic acids so as to facilitate the function of double stranded interfering RNA. Such analogs comprise, for examnple, phosphoramide (Beaucage et al., Tetrahedron 49(10):1925 1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 (1986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook;

Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference.

The nucleic acids may be single stranded or double stranded, as specified, or form both double stranded and single stranded regions. The nucleic acid may be DNA, RNA or a hybrid, where the nucleic acid contains any combination of deoxyribo- and ribonucleotides, and any combination of bases, including uracil, adenine, thymine, cytosine, guanine, inosine, xathanine hypoxathanine, isocytosine, isoguanine, etc. As used herein, the term “nucleotide” includes naturally occurring, and modified nucleotides.

The terms used to describe sequence relationships between two or more nucleotide sequences include “identical,” “selected from,” “substantially identical,” “complementary,” and “substantially complementary.”

A subject nucleic acid sequence is “identical” to a reference sequence if the two sequences are the same when aligned for maximum correspondence over the length of the nucleic acid sequence or a region thereof.

“Complementary” refers to the topological compatibility or matching together of interacting surfaces of two nucleic acid sequences. Thus, the two molecules can be described as complementary, and furthermore, the contact surface characteristics are complementary to each other. A first sequence is complementary to a second sequence if the nucleotides of the first sequence have the sequence of the nucleotides in the sequence binding partner of the second sequence. Thus, the sequence whose sequence 5′-TATAC-3′ is complementary to a sequence whose sequence is 5′-GTATA-3′.

A nucleic acid sequence is “substantially complementary” to a reference nucleotide sequence if the sequence complementary to the subject nucleotide sequence is substantially identical to the reference nucleotide sequence.

“Specifically binds to” refers to the ability of one molecule, typically a molecule such as a nucleic acid, to contact and associate with another specific molecule even in the presence of many other diverse molecules. For example, a single-stranded RNA can “specifically bind to” a single-stranded RNA that is complementary in sequence.

A nucleic acid sequence “specifically hybridizes” to a target sequence if the sequence hybridizes to the target under stringent conditions. “Stringent conditions” refers to temperature and ionic conditions used in nucleic acid hybridization. Stringent conditions depend upon the various components present during hybridization. Generally, stringent conditions are selected to be about 10° C., and preferably about 5° C. lower than the thermal melting point (Tm) for the specific sequence at a defined ionic strength and pH. The Tm is the temperature (under defined ionic strength and pH) at which 50% of a target sequence hybridizes to a complementary polynucleotide.

A first sequence is an “antisense sequence” with respect to a second sequence if a polynucleotide whose sequence is the first sequence specifically hybridizes with a polynucleotide whose sequence is the second sequence.

“Substantially pure” means an object molecule is the predominant molecule present (i.e., on a molar basis, more abundant than any other individual macromolecular species in the composition), and a substantially purified fraction is a composition wherein the object molecule comprises at least about 50% (on a molar basis) of all molecular species present. Generally, a substantially pure composition means that about 80 to 90% or more of the macromolecular species present in the composition is the purified species of interest. The object molecule is purified to essential homogeneity (contaminant molecules cannot be detected in the composition by conventional detection methods) if the composition consists essentially of a single macromolecular species. Solvent molecules, small molecules (<500 Daltons), stabilizers (e.g., BSA), and elemental ion molecules are not considered macromolecular species for purposes of this definition.

“Telomerase activity” refers to the synthesis of telomeres by telomerase. Measurement of telomerase activity is preferably by an assay called TRAP (Telomeric Repeat Amplification Protocol) (Kim, et al., Science 266:2011-2015 (1994); Piatyszek, et al., Meth Cell Sci 17:1-15 (1995); Wright, et al., Nucl Acids Res. 23:3794-3795 (1995)). The TRAP assay has two phases, but can be performed in a single tube. In the first phase, an unlabelled oligonucleotide primer is extended by the telomerase activity in the cell extract being assayed, using labeled deoxynucleotide triphosphates. In the second phase, the products of the first phase are amplified, using the polymerase chain reaction. The amplification products are then analyzed by electrophoresis, revealing a series of bands differing in length by six nucleotides. Activity can be conveniently quantitated by phosphorimaging. The TRAP assay requires only 100 to 1000 cells.

More recently a modification of the TRAP assay called TRAPeze™ telomerase assay kit (Oncor); (Feng et al., supra). A modified reverse primer sequence eliminates the need for a wax barrier and for a hot start, reduces amplification artifacts, and permits better estimation of telomerase processivity. Further, a template and a corresponding primer are used as an internal standard to improve linearity and detect inhibitors of amplification (Holt, et al., Meth Cell Science 18:237-248 (1996)). Whereas in the TRAP assay a nucleotide triphosphate is labeled, in the TRAPeze™ assay the primer is labeled.

“Telomerase-related condition” refers to a condition in a subject maintained by telomerase activity within cells of the individual. Telomerase-related conditions include, e.g., cancer (telomerase-activity in malignant cells), fertility (telomerase activity in germ-line cells) and hematopoiesis (telomerase activity in hematopoietic stem cells).

This invention provides methods of treating conditions in mammals involving undesirable expression of telomerase activity. The methods involve administering to the subject an amount of interfering nucleic acid of this invention effective to interfere with telomerase activity i.e. a pharmacologically effective amount. Cells that express telomersase activity and that can be targets of telomerase RNA interference therapy include telomerase expressing cancer cells, germ-line cells and telomerase expressing hematopoietic cells. Interfering with telomerase activity is also useful in treating veterinary proliferative diseases. Because telomerase is active in a limited number of cell types, e.g. tumor cells, germline cells, certain stem cells of the hematopic system, T and B cells, sun-damaged skin, and proliferative cervix, most normal cells are not affected by telomerase RNA interference therapy. Steps can also be taken to avoid contact of the interfering nucleic acid with germline or stem cells, if desired, although this may not be essential.

The interference of telomerase activity in telomerase-expressing cancer cells results in eventual cell crisis and senescence. Interfering nucleic acids are expected to be useful in treating cancer. The types of cancer that can be treated include, for example, cancer of the breast, prostate, lung, and colon, as well as lymphocytic and myeloid leukemias, melanoma, hepatoma, and neuroblastoma.

Germline cells, i.e., oocytes and sperm, express telomerase activity. Therefore, interference of telomerase activity in germ-line cells is useful for methods of contraception or sterilization.

A subpopulation of normal hemopoetic cells, e.g., B and T cells, and hematopoietic stem cells express telomerase. Therefore, interference of telomerase in such cells is useful for immunosuppression and for selectively down-regulating specific branches of the immune system, e.g. a specific subset of T cells. Such method are useful in anti-inflammatory therapies. Interference of telomerase activity in certain lines of cells using interfering nucleic acids is attractive because after therapeutic effect, the treatment can be halted and stem cells will repopulate the system with healthy cells. Eukaryotic organisms that express telomerase, e.g. yeast, parasites, and fungi, can infect the body. Such infections can be treated by interfering with telomerase activity in these organisms, thereby halting growth of the organism.

“Pharmaceutical composition” refers to a composition suitable for pharmaceutical use in a mammal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences (19^{th} ed., 1995). Preferred pharmaceutical carriers depend upon the intended mode of administration of the active agent. Typical modes of administration include enteral (e.g., oral) or parenteral (e.g., subcutaneous intramuscular, or intravenous intraperitoneal injection; or topical, transdermal, or transmucosal administration).

Interfering nucleic acids can be delivered conveniently in the form of a pharmaceutical composition comprising a pharmaceutically acceptable carrier and a pharmacologically effective amount of the agent. The pharmaceutical composition can be administered by any means known in the art for delivery of such molecules. However, systematic administration by injection is preferred. This includes intratumoral, intramuscular, intravenous, intraperitoneal, and subcutaneous injection. The pharmaceutical compositions, can be administered in a variety of unit dosage forms depending upon the method of administration. For example, unit dosage forms for perenteral administration include unit doses of injectable solutions.

The form, amounts and timing of administration generally are a matter for determination. In one embodiment, the pharmaceutical composition is a composition delivered as a unit dosage form to provide a systemic or local concentration of 50-100 nM although other concentrations may be used, based on experimental results. Two dsRNA molecules per cell are sufficient to initiate the degradative process (Fire, et al., Nature 391:806-811 (1998)).

A striking feature of the phenomenon is its sequence specificity; the sequence of the antisense strand is especially crucial (Parrish, et al., Molecular Cell 6:1077-1087 (2000)). Double stranded RNA is a more effective inhibitory agent than is antisense alone in many systems (Waterhouse, et al., Proc Natl Acad Sci USA 95:13959-13964 (1998)).

Several other methods for introduction or uptake of interfering nucleic acids into a cell are well known in the art. These methods include but are not limited to, retroviral infection, adenoviral infection, transformation with plasmids, transformation with liposomes containing interfering nucleic acid, biolistic nucleic acid delivery (i.e. loading the nucleic acid onto gold or other metal particles and shooting or injecting into the cells), adeno-associated virus infection and Epstein-Barr virus infection. These may all be considered “expression vectors” for the purposes of the invention.

For delivery into cells, recombinant production of interfering nucleic acids through the use of expression vectors is particularly useful. Accordingly, the invention also provides expression vectors, e.g., recombinant nucleic acid molecules comprising expression control sequences operatively linked to the nucleotide sequence encoding the interfering nucleic acids. Expression vectors can be adapted for function in prokaryotes or eukaryotes (e.g., bacterial, mammalian, yeast, Aspergillus, and insect cells) by inclusion of appropriate promoters, replication sequences, markers, etc. for transcription of RNA including mRNA. The construction of expression vectors and the expression thereof in transfected cells involves the use of molecular cloning techniques also well known in the art (Sambrook et at., Molecular Cloning—A Laboratory Manual (2nd ed. 1989); Ausubel et al., Current Protocols in Molecular Biology). Useful promoters for such purposes include a metallothionein promoter, a constitutive adenovirus major late promoter, a dexamethasone-inducible MMTV promoter, a SV40 promoter, a MRP pol III promoter, a constitutive MPSV promoter, a tetracycline-inducible CMV promoter (such as the human immediate-early CMV promoter), a constitutive CMV promoter, and EF-1alpha. Recombinant DNA expression plasmids can also be used to prepare the interfering nucleic acids of the invention for delivery by means other than by gene therapy, although it may be more economical to make short oligonucleotides by in vitro chemical synthesis.

Methods of transfecting nucleic acids into manunalian cells and obtaining their expression for in vitro use or for gene therapy, are well known to the art (see, e.g., Methods in Enzymology, vol. 185 (Goeddel, ed. 1990); Krieger, Gene Transfer and Expression—A Laboratory Manual (1990)). Cells can be transfected with plasmid vectors, for example, by electroporation. Cells can be transfected with nucleic acids by calcium phosphate precipitation, DNA liposome complexes, by particle-mediated nucleic acids transfer (biolistics) or with liposomes.

A variety of expression vectors may be utilized to express interfering RNA. The expression vectors are constructed to be compatible with the host cell type. Expression vectors may comprise self-replicating extrachromosomal vectors, e.g., for cloning vectors, or vectors which integrate into a host genome.

A preferred mammalian expression vector system is a retroviral vector system such as is generally described in Mann et al., Cell 33:153-9 (1993); Pear et al., Proc. Natl. Acad. Sci. U.S.A. 90(18):8392-6 (1993); Kitamura et al., Proc. Natl. Acad. Sci. U.S.A, 92:9146-50 (1995); Kinsella et al., Human Gene Therapy, 7:1405-13; Hofmann et al., Proc. Natl. Acad. Sci. U.S.A., 93:5185-90; Choate et al., Human Gene Therapy 7:2247 (1996); PCT/US97/01019 and PCT/US97/01048, and references cited therein, all of which are hereby expressly incorporated by reference.

Post-transcriptional gene silencing, i.e. double stranded RNA interference, appears to be a phenomenon ranging widely across kingdoms of plants, fungi, invertebrates, and vertebrates and exhibiting many common features (Cogoni, et al., Curr Opinion Genet Devel 10:638-643 (2000)). The first evidence for dsRNA in mammals was reported by Wianny and Zernick-Goetz (Wianny, et al., Cell Biol 2:70-75 (2000)) who showed that dsRNA is effective as a specific inhibitor of three genes in early mouse development. dsRNA specifically reduces dormant maternal mRNAs in mouse oocytes and is more effective than antisense RNA (Svoboda, et al., Development 127:4147-4156 (2000)). It has been recently been reported that dsRNA is processed into short oligonucleotides (22mers) and specifically inhibits translation of the corresponding mRNA in a variety of human cell lines (Hammond, et al., Nature 404:293-296 (2000)).

Double stranded RNA is known to induce a variety of genes as a defense against viral infection. These include protein kinase PKR, interleukin 1 and 6, 2′,5′-oligoadenylate synthetase, interferon regulatory factor IRF-1, intracellular adhesion molecule ICAM-1, vascular cell adhesion molecule ICAM-1, and E-selectin (Harcourt, et al., J Interferon Cytokine Res 20:1007-1013 (2000)). Double stranded RNA binds to inactive protein kinase PKR and activates its kinase activity. This kinase activity phosphorylates eIF-alpha and blocks protein synthesis. Thus, from the point of view of using dsRNA as an anticancer agent, induction of the interferon response is an undesirable effect.

To be valuable therapeutically, a telomerase inhibitor should cause a reduction in telomere length leading to cell death. Telomere length can be measured by flow cytometry using a telomeric probe (CCCTAA)₃ of peptide nucleic acid (PNA). The PNA binds to DNA more tightly than does complementary DNA or RNA. The fluorochrome to be conjugated is FAM (carboxyfluorescein succinimidyl ester).

Telomerase inhibition should not inhibit cell division until telomeres have shortened to a critical length. The longer the telomeres initially, the greater the delay expected in inhibition of cell division. Viable cell number will be quantitated by staining with trypan blue.

Telomere erosion leads to death by apoptosis. Adequate telomere length apparently inhibits two apoptosis execution mechanisms, induction of nuclear calcium-dependent endonucleases and activation of the caspase family of death proteases (Herbert, et al., Proc Nat Acad Sci 96:14276-14281 (1999)). Kondo et al. (Shammas, et al., Oncogene 18:6191-6200 (1999)) reported that transfection of antisense to human telomerase RNA into human malignant glioma cells caused expression of a high level of interleukin-1 beta-converting enzyme (ICE, a cysteine protease) and apoptosis. Apoptosis in telomerase inhibition-treated cells can be measured quantitatively by terminal deoxynucleotidyl transferase binding and flow cytometry (Lee, et al., Proc Natl Acad Sci 98:3381-3386 (2001); Gupta, et al., J. Natl Canc Inst 88:1095-1096 (1996); Bryan, et al., EMBO J. 14:4240-4248 (1995); Lundblad, et al., Cell 73:347-360 (1993)).

### EXAMPLE 1

### Telomerase Inhibition by Small Interfering RNAs in Mammalian Cells

Inhibition of telomerase in cancer cells leads to telomere shortening, end-to-end chromosomal fusion, and apoptosis. Hence it is an attractive target for the development of anticancer agents. We have explored the utility of small interfering RNAs to inhibit telomerase in mammalian cells. We designed a 21-nucleotide duplex RNA (dsRNA) targeting the template region of human telomerase RNA. See Seq. ID No. 1.

r(UUG UCU AAC CCU AAC UGA G)d(TT)
d(TT)r(AAC AGA UUG OGA UUG ACU C)

Human cervical carcinoma cells (HeLa) or human fibrosarcoma cells (HT-1080) were plated in 24-well plates. The following day, while the cells were still subconfluent, dsRNA (Xeragen) was introduced in Optimem medium without lipid or serum and 6 hours later replaced with serum-containing medium. Cells were harvested after an additional 40 hours and analyzed for telomerase activity using the TRAPeze™ assay (Intergen). The more duplex added, the greater the inhibition. Significant inhibition was observed at a low extracellular concentration; I.C.50 was ˜100 nM for each cell line. Inhibition of telomerase activity in human cancer cells using small interfering RNAs warrants further exploration.

### EXAMPLE 2

siRNAs

Double stranded RNA was synthesized by Xeragon (Huntsville Ala.). For telomerase RNA, hTR #1 siRNA targeted the region containing the telomere repeat template sequence, shown in boldface:

5′-UUGU CUA ACC CUA ACU GAG-TT-3′
3′-TT-AACA GAU UGG GAU UGA CUC-5

hTR #2 siRNA targeted a 19 bp sequence centered in the 26 bp L6 loop, the longest single stranded region in hTR according to the secondary structure:

5′-GGCT TCT CCG GAG GCA CCC TT-3′
3′-TT-CCGA AGA GGC CTC CGT GGG-5′

In the case of the mRNA for telomerase's protein catalytic subunit, hTERT, the target was the region containing the site of the dominant negative mutation (bolded) used to inactivate the gene:

5′-CAAG GUG GAU GUG ACG GGC TT-3′
3′-TT-GUUG CAC CUA CAC UGC CCG-5′

siRNA Transfection

Cell lines were obtained from American Type Culture Collection and maintained in the media recommended by them. Cells were transfected. Cells in 0.5 ml aliquots were plated in a 24-well plate at a concentration estimated to provide 30-40% confluence 16 h later. At that time, dsRNA for either human telomerase RNA or human telomerase reverse transcriptase (0.25, 0.5, 1, or 2 μg) was diluted with 125 μl of Optimem medium (Invitrogen). In a separate tube, 7.5 μl Oligofectamine (Invitrogen) was diluted with 30 μl of Optimem. The two solutions were mixed gently by inversion and incubated at room temperature for 7-10 min. The contents of the two tubes were then combined, mixed gently by inversion, and incubated at room temperature for 20-25 min. 100 μl containing the liposome complexes was added to the culture medium in each well and mixed by gentle rocking for 30 sec. HeLa cells were maintained in serum throughout, but, for other cell types, serum was removed for the first four hours of transfection. At 22 or 42 hours, cells were trypsinized, counted, and 2000 cells removed for assay of telomerase activity.

Telomerase Activity

Telomerase activity was measured by the TRAPeze assay (Serologicals, formerly Intergen).

Quantitation of Telomerase RNA

Total RNA was purified using the SV Total RNA Isolation System (Promega). Telomerase RNA was quantified by a reverse-transcriptase-polymerase chain reaction (RT-PCR) assay. 50 or 100 ng of total RNA was incubated in 5 μM random hexamers (Pharmacia-LKB), 0.5 mM deoxyribonucleotide triphosphates×4, 0.5 unit/μl RNAsin (Promega), 1 mM dithiothreitol, and 2.5 units/ml Moloney leukemia virus reverse transcriptase (Invitrogen) in 50 mM KCl, 10 mM Tris-Cl, pH 9.0, and 0.1% Triton X-100 in 20 μl for 45 min at 37. The reaction was then heated to 95° for 10 min to denature the reverse transcriptase.

Each PCR reaction contained 10 μl of the reverse transcriptase reaction mixture, 0.5 μM primers, 10 mM deoxyribonucleotide triphosphates×4, 2.5 μCi [alpha-³²P]dCTP, 3000 Ci/mmol, in 2.0 mM MgCl₂, 40 mM KCl, 8 mM Tris-Cl, pH 9.0, and 0.1% Triton X-100 in 50 μl. The products of the PCR reaction were electrophoresed in 10% nondenaturing polyacrylamide gel in 1×TBE at 40 V for 18 h. Radioactivity was quantified by phosphorimaging. The value of the no RNA control was subtracted from each experimental value.

The primers used were 5′-CTG GGA GGG GTG GTG GCC ATT T-3′ and 5′-CGA ACG GGC CAG CAG CTG ACA T-3′. Reaction parameters were 94° for 20 sec, 50° for 20 sec, and 72° for 30 sec for 25 cycles.

Quantitation of hTERT mRNA

hTERT mRNA was quantified by an RT-PCR method similar to that used to quantify telomerase RNA except that the Mg++ concentration in the PCR reaction was

1.0 mM and the primers were 5′-GCC AGA ACG TTC CGC AGA GAA AA-3′ and

5′-AAT CAT CCA CCA AAC GCA GGA GC-3′. Reaction parameters were 94° for 20 sec, 48° for 30 sec, and 72° for 30 sec. for 30 cycles.

Effect of siRNAs

On Telomerase Activity

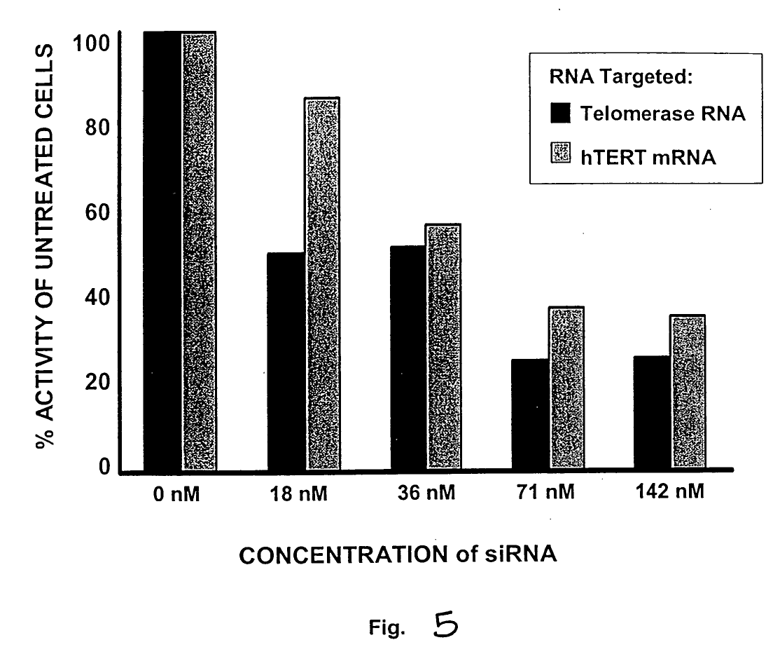

SiRNAs for hTR and hTERT depressed the telomerase activity of HCT-15 human colon carcinoma cells in a dose-dependent manner. FIG. 5 shows the effect at 44 h. Results throughout are reported as a percentage of telomerase activity of cells treated with the lipid transfecting agent only (i.e. as a percentage of activity of “untreated” cells). The maximum effect observed with HCT-15 cells was 25% of untreated cell activity for hTR siRNA and 35% of untreated cell activity for hTERT siRNA.

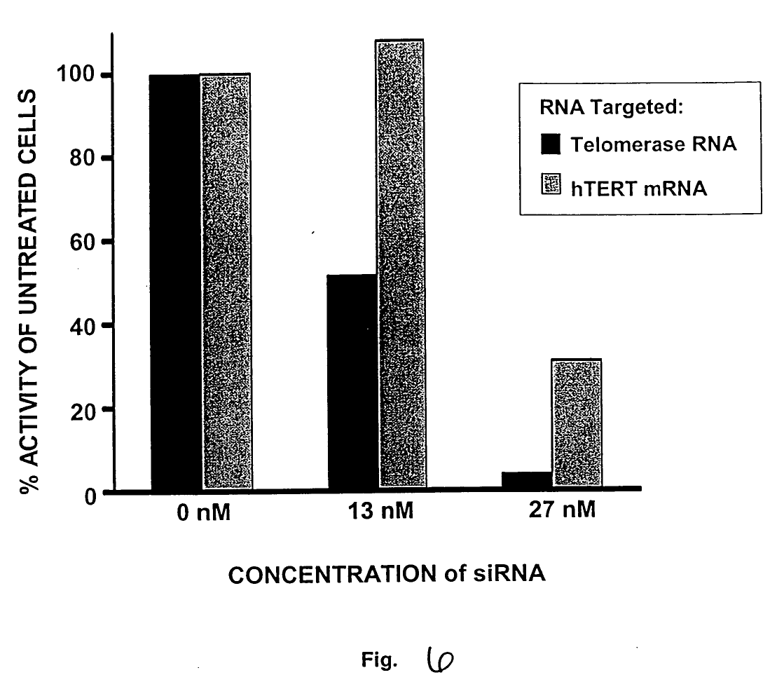

Both agents depressed telomerase activity also in HeLa human cervical carcinoma cells in a dose-dependent manner. FIG. 6 shows the effect at 42 h. In both cell types, the siRNA for hTR was more inhibitory than the siRNA for hTERT at a given concentration. In dose-response experiments of similar design, telomerase inhibition was seen also with other types of carcinoma cells, viz. NCI H23 human lung carcinoma cells and A431 human epidermoid carcinoma cells.

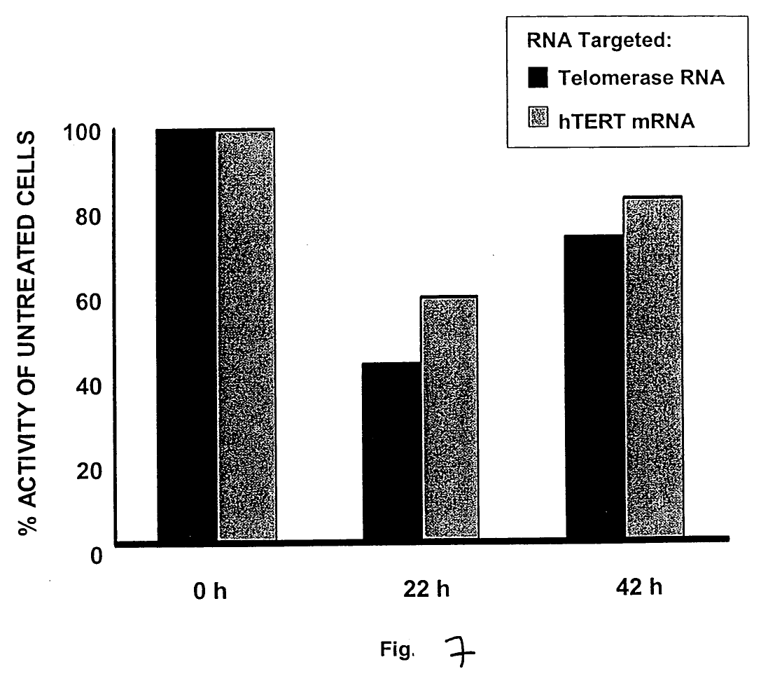

Each agent depressed telomerase activity also in cells of mesodermal origin, viz. HT-1080 human fibrosarcoma cells (FIG. 7) and CCL121 human osteosarcoma. However in both these cell lines, inhibition was greater at 22 h than at 46 hours, unlike the results of the carcinoma cell lines tested.

Using HeLa cells, four strategies were used in an effort to demonstrate more complete inhibition of telomerase activity. First, cells were treated with higher concentrations of siRNA for hTR, up to 1136 nM, but inhibition was not further increased.

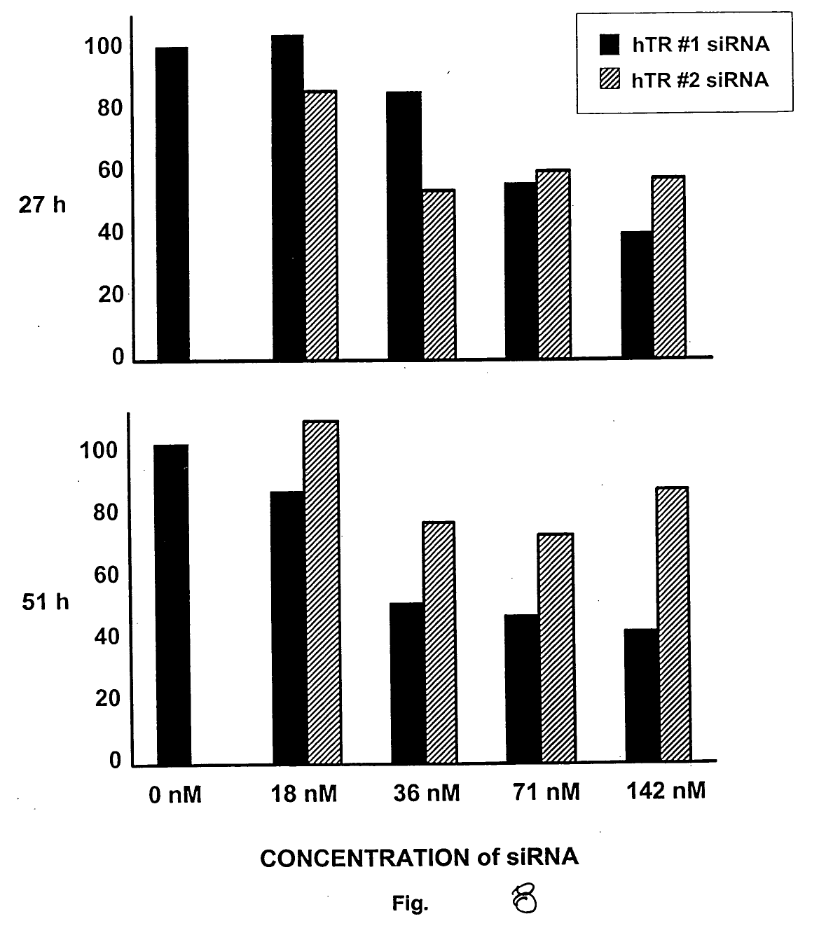

Second, cells were treated with siRNA for hTR on a daily basis. FIG. 8A shows the results of treatment using various concentrations. The bars marked 1, 2, and 3 represent the telomerase activity after one, two, and three days of treatment, each assayed 24 h after the last dose. There was progressive inhibition for the 72 h period investigated. However the lowest value reached was only 35% of the untreated. Additions of 142 nM did not produce appreciably more inhibition than those of 71 nM. To address the question as to whether multiple transfections decrease the telomerase activity more than a single initial transfection, cells were transfected either at 0 h only or at both 0 and 24 h and both sets were assayed at 48 h. As shown in FIG. 8B, two transfections resulted in lower telomerase activity than a single one.

Third, cells were treated with siRNAs for both hTR and hTERT simultaneously. However inhibition did not exceed that seen with each separately.

Fourth, cells were treated with siRNA targeting hTR, but at a different site. On the assumption that internally hybridized regions would not be accessible to siRNAs, we chose a 19 bp sequence centered in the 26 bp L6 loop, the longest single stranded region of the hTR secondary structure proposed by Chen et al. However, at 51 h, this second generation siRNA for hTR was less inhibitory than the first (FIG. 9).

On Telomerase RNA Content.

The effect of siRNAs on telomerase RNA content is shown in FIG. 10. Compared to HeLa cells treated with the lipid transfecting agent Oligofectamine alone, cells treated with hTR siRNA had decreased telomerase RNA content in the RT-PCR assay by over 50%. In contrast, cells treated with hTERT siRNA had no decrease in telomerase RNA.

All references are incorporated herein by reference.

1. A nucleic acid comprising sense and anti-sense nucleic acids, wherein said sense and anti-sense nucleic acids are substantially complementary to each other and are capable of forming a double stranded nucleic acid and wherein one of said sense or anti-sense nucleic acids is substantially complementary to a target nucleic acid comprising telomerase RNA or mRNA encoding telomerase reverse transcriptase (TERT).

2. A nucleic acid according to claim 1 wherein said telomerase is human telomerase.

3. The nucleic acid of claim 1 wherein said sense and anti-sense nucleic acids comprise RNA.

4. The nucleic acid of claim 3 wherein said sense and anti-sense RNAs are in the form of a double stranded interfering RNA.

5. The nucleic acid of claim 4 wherein said interfering RNA is a double stranded short interfering RNA (siRNA).

6. The nucleic acid of claim 5 wherein said siRNA comprises a double stranded interfering RNA of less than about 30 nucleotides.

7. The nucleic acid of claim 5 wherein said siRNA targets telomerase RNA.

8. The nucleic acid of claim 7 wherein said siRNA targets the telomerase template sequence.

9. The nucleic acid of claim 8 wherein said telomerase template sequence comprises CUAACCCUAAC (SEQ ID NO:1).

10. The nucleic acid of claim 7 comprising the double stranded nucleic acid

| | |
|---|---|
| 5′-UUGU CUA ACC CUA ACU GAG-TT-3′ | (SEQ ID NO:6) |
| 3′-TT-AACA GAU UGG GAU UGA CUC-5′. | (SEQ ID NO:7) |

11. The nucleic acid of claim 7 comprising the double stranded sequence

| | |
|---|---|
| 5′-GGCT TCT CCG GAG GCA CCC-TT-3′ | (SEQ ID NO:8) |
| 3′-TT-CCGA AGA GGC CTC CGT GGG-5′. | (SEQ ID NO:9) |

12. The nucleic acid of claim 5 wherein said siRNA targets TERT mRNA.

13. The nucleic acid of claim 12 comprising the double stranded sequence
(SEQ ID NO:10)
5′-CAAG GUG GAU GUG ACG GGC TT-3′
(SEQ ID NO:11)
3′-TT-GUUG CAC CUA CAC UGC CCG-5′.

14. An expression vector comprising the nucleic acid of claim 1.

15. A method for inhibiting telomerase activity comprising contacting a telomerase expressing cell with a nucleic acid according to claim 1.

16. A method for inhibiting telomerase activity comprising contacting a telomerase expressing cell with the expressing vector of claim 14.

17. The method of claim 15 wherein said cell comprises a cancer cell.

18. A pharmaceutical composition comprising the nucleic acid according to claim 1 and a pharmaceutically acceptable carrier.

19. A pharmaceutical composition comprising the expression vector of claim 14 and a pharmaceutically acceptable carrier.

20. The method of claim 16 wherein said cell comprises a cancer cell.
